(19) United States
(12) Patent Application Publication
(10) Pub. No.: US 2013/0296800 A1
(43) Pub. Date: Nov. 7, 2013

(54) SYRINGE PROVIDED WITH NEEDLE, AND METHOD FOR PRODUCING SYRINGE PROVIDED WITH NEEDLE

(71) Applicant: Manabu OE, Nakakoma-gun, Yamanashi (JP)

(72) Inventors: Masafumi Takemoto, Fujinomiya-shi (JP); Manabu Oe, Nakakoma-gun (JP)

(73) Assignee: TERUMO KABUSHIKI KAISHA, Shibuya-ku, Tokyo (JP)

(21) Appl. No.: 13/980,693

(22) PCT Filed: Nov. 25, 2011
(86) PCT No.: PCT/JP2011/077219

§ 371 Date: Jul. 19, 2013

(30) Foreign Application Priority Data
Jan. 19, 2011 (JP) 2011008440

(51) Int. Cl. A61M 5/34

(52) U.S. Cl. CPC A61M 5/34
USPC 604/240; 156/73.1

(57) ABSTRACT

A syringe is provided with a needle that includes a needle tube having a needle tip that can puncture a living body, a needle holding member, and an outer tube that is filled with a drug. The needle holding member is formed integrally and coaxially with the needle tube by insert molding. The needle holding member has a distal end and a proximal end, and can hold the needle tube in a state where the needle tip of the needle tube is protruded from the distal end. The outer tube is fixed to the needle holding member by welding. The needle holding member and the outer tube are formed from essentially the same material.

This application is the national phase entry under U.S.C. §371 of International Application No. PCT/JP2011/077219, filed on Nov. 25, 2011, which claims priority under 35 U.S.C. §119 to Japanese Patent Application No. JP2011-008440, filed on Jan. 19, 2011, the contents of which are hereby incorporated by reference in their entirety for all purposes.

### TECHNICAL FIELD

The present invention relates to a syringe provided with a needle in which a syringe is formed on a needle tube by insert molding, and a method for producing a syringe provided with a needle.

### BACKGROUND OF THE INVENTION

In general, a syringe provided with a needle is composed of a needle tube which is provided at a distal portion thereof with a needle tip capable of puncturing a living body, and a needle hub which holds the needle tube with the needle tip of the needle tube being protruded. In addition, the syringe provided with a needle is configured by connecting a syringe to the needle hub. Hitherto, the needle tube has been fixed to the needle hub by use of an adhesive, in the state of being inserted and passed in an insertion hole in the needle hub. See, for example, Japanese Patent Laid-open No. 2006-116163 (hereinafter Patent Document 1).

In recent years, a syringe provided with a needle has been proposed in which a needle hub is eliminated and a needle tube is fixed directly to a syringe to be filled with a drug. In this syringe provided with a needle, also, the needle hub is fixed to the syringe by use of an adhesive, like in the case of the related-art syringe provided with a needle.

However, the syringe is filled with a drug in some cases. Therefore, there is a risk of contact (liquid contact) of an adhesive, used to fix the needle tube and the syringe to each other, with the drug filled in the syringe, leading to the problem that the drug is adversely affected. In recent years, therefore, there has been a need for a technology of integrally forming a needle tube and a syringe by insert molding, as a fixing method in which no adhesive is used.

In addition, in the method of forming the needle tube and the syringe at a time by insert molding, not only the mold for forming the syringe would become larger in size but also complicated parting lines of mold are involved for preventing the needle tube from being damaged. As a result, a large-sized mold with a complicated structure is required. Thus, there is a problem that the steps for producing the syringe provided with a needle become complicated.

Furthermore, a core pin for forming a tube hole of the syringe at the time of insert molding has to be provided with a portion for supporting the needle tube. After molding, therefore, the tube hole is formed therein with a space at the part where the needle tube has been supported. The space finishes playing a role when the insert molding is over. After the insert molding, therefore, the space does not fulfill any special function and is unnecessary. As a result, the space becomes a dead volume, in which the drug would be left uninjected.

In consideration of the above-mentioned problems, it is an object of the present invention to provide a syringe provided with a needle such that a needle tube and a syringe can be integrally formed by insert molding conducted without using any adhesive and simplification of a mold is achieved, and a method for producing the syringe provided with a needle.

### SUMMARY OF THE INVENTION

In order to solve the above-mentioned problems and to attain the object of the present invention, a syringe is provided with a needle according to the embodiments of present invention. The syringe includes a needle tube and a needle tube holding member. The needle tube has a needle tip capable of puncturing a living body. The needle holding member is formed integrally and coaxially with the needle tube by insert molding, has a distal end and a proximal end, and holds the needle tube with a needle tip of the needle tube protruding from the distal end. The syringe provided with a needle is provided also with an outer tube having one end portion to which the proximal end of the needle holding member is fixed by welding, and other end portion located on the side axial-directionally opposite to the one end portion and provided with an opening, the outer tube being to be filled with a drug. Besides, the needle holding member and the outer tube are formed from essentially the same material.

In addition, a method for producing a syringe provided with a needle according to the present invention includes the following steps:
forming an outer tube to be filled with a drug;
forming a needle tube having a needle tip capable of puncturing a living body, integrally with a needle holding member which holds the needle tube, by insert molding; and
fixing the needle holding member formed integrally with the needle tube to the outer tube by welding, wherein the needle holding member and the outer tube are formed from essentially the same material.

According to the method for producing a syringe provided with a needle of the present invention, the outer tube to be filled with a drug and the needle holding member which holds the needle tube are formed separately. This ensures that the mold for insert molding can be reduced in size, and a simplified mold can be contrived. In addition, with the needle tube and the needle holding member being integrally formed by insert molding, the needle tube can be fixed without using any adhesive. Furthermore, since the outer tube and the needle holding member are formed from the same material and joined to each other, good bonding properties between the outer tube and the needle holding member can be obtained.

### BRIEF DESCRIPTION OF DRAWINGS

FIG. 1 is a perspective view showing an embodiment of a syringe provided with a needle according to the present invention.

FIG. 2 is a perspective view showing an essential part of the embodiment of the syringe provided with a needle according to the present invention.

FIG. 3 is a sectional view showing an outer tube according to the embodiment of the syringe with a needle of the present invention.

FIGS. 4A to 4C illustrate a method for producing the embodiment of the syringe provided with a needle according to the present invention, wherein FIG. 4A is a sectional view of a needle tube, FIG. 4B is a sectional view showing the state where the needle tube and a needle holding member are united, and FIG. 4C is a sectional view showing the state where the holding member which holds the needle tube and an outer tube are integrally fixed.

FIG. 5 is a perspective view showing the state where a cap is mounted to the embodiment of the syringe provided with a needle according to the present invention.

FIG. 6 is a sectional view of the syringe provided with a needle and the cap which are shown in FIG. 5.

### DETAILED DESCRIPTION OF THE INVENTION

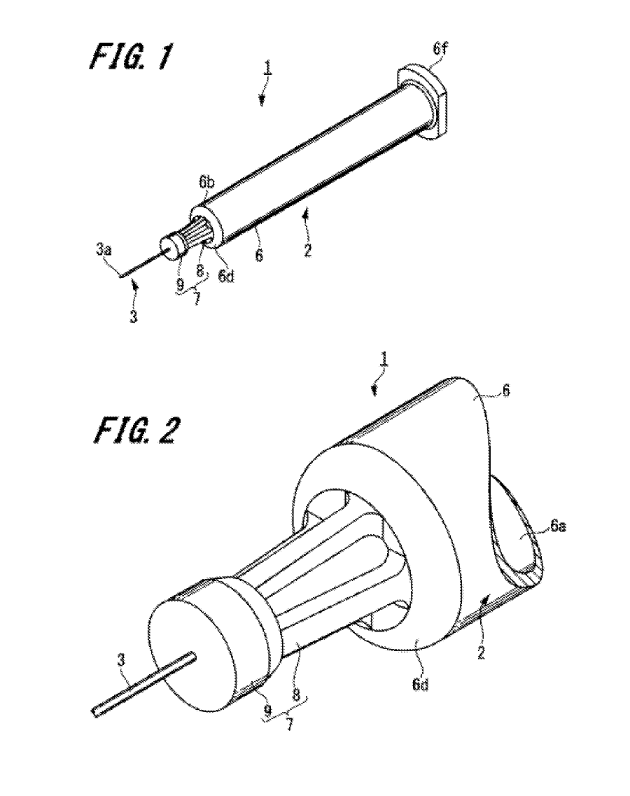

With reference to FIGS. 1 to 6, an exemplary embodiment of a syringe including a needle according to the embodiments of the present invention will be described below. Incidentally, members shown in common in the drawings are denoted by the same reference numerals. In addition, the present invention is not restricted to the following mode.

Incidentally, description will be made in the following order:

1) Configuration Example of Syringe Provided with Needle

2) Assemblage of Syringe Provided with Needle

Configuration Example of Syringe Provided with Needle

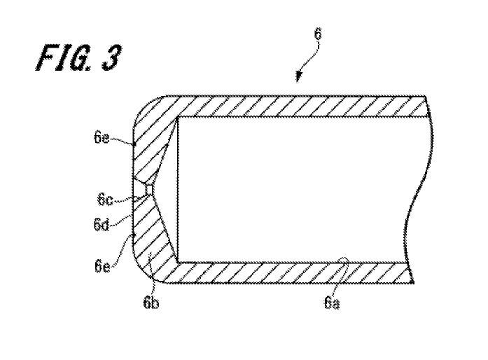

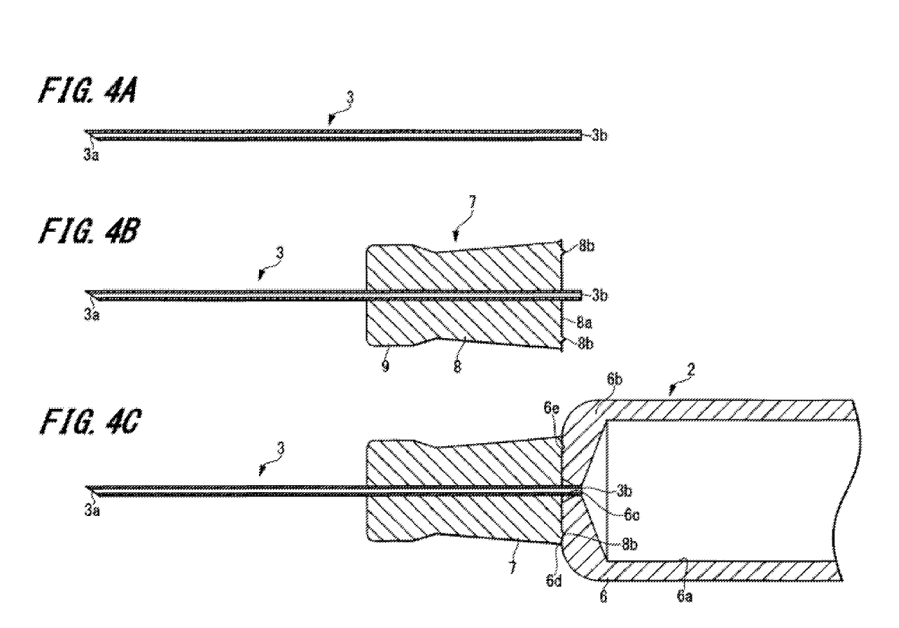

FIG. 1 is a perspective view showing the syringe provided with a needle according to the present embodiment, and FIG. 2 is a perspective view showing an essential part of the syringe provided with a needle according to the present embodiment. FIG. 3 is a sectional view showing an outer tube of the syringe provided with a needle according to the present embodiment, and FIGS. 4A to 4C illustrate a method for producing the syringe provided with a needle according to the present embodiment.

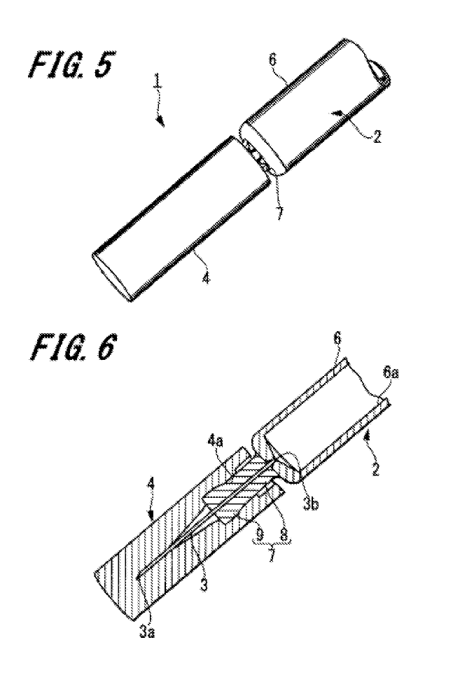

The syringe 1 provided with a needle is used for puncturing a surface of a skin with a needle tip and injecting a drug into a living body. As shown in FIG. 1, this syringe 1 provided with a needle includes a syringe 2 to be filled with a drug, and a needle tube 3 fixed to the syringe 2. In addition, a cap 4 (see FIGS. 5 and 6) is mounted to the syringe 1 provided with a needle.

### Needle Tube

In the following, the needle tube 3 will be first described. As the needle tube 3, one which has a size of 10 to 33 gauge (outside diameter: 3.5 to 0.2 mm), preferably 16 to 33 gauge (outside diameter 1.7 to 0.2 mm), according to the ISO medical needle tube standard (ISO 9626: 1991/Amd. 1:2001(E)), is used. As shown in FIG. 4A, the needle tube 3 is formed at one axial-directional end thereof with a cutting edge surface for making a needle tip 3a have an acute angle. The needle tip 3a on one side in the axial direction of the needle tube 3 is made to puncture a living body.

Examples of the material for the needle tube 3 include stainless steel, but this is not restrictive. Other metals such as aluminum, aluminum alloys, titanium, and titanium alloys can also be used. In addition, the needle tube 3 is not restricted to a straight needle but may be a tapered needle of which at least a part is in a tapered shape. It suffices that the tapered needle is greater in diameter at a proximal portion thereof than at an end portion of the needle tip 3a and that an intermediate portion of the needle has a tapered structure. Besides, the sectional shape of the needle tube 3 is not restricted to a circle but may be a polygon such as a triangle.

Furthermore, a surface on the needle tip 3a side of the needle tube 3 is coated with a coating agent such as silicone rubber or fluoro-resin. This ensures that when the needle tube 3 punctures a living body, the friction between the skin and the needle tube can be reduced, whereby the pain attendant on the puncture can be alleviated.

The needle tube 3 is fixed to the syringe 2, with the needle tip 3a protruding to the outside. As shown in FIG. 4B, the needle tip 3a on one side in the axial direction of the needle tube 3 protrudes from a distal portion of a needle holding member 7 of the syringe 2 which will be described later. A proximal section 3b on the other side in the axial direction, namely, on the side opposite to the needle tip 3a, protrudes from a proximal portion of the needle holding member 7.

### Syringe

Now, the syringe 2 will be described below. The syringe 2 is provided with an outer tube 6 to be filled with a drug, and the needle holding member 7 which holds the needle tube 3. As shown in FIG. 3, the outer tube 6 is formed in a substantially cylindrical shape. The needle holding member 7 is fixed to one end portion 6b on one side in the axial direction of the outer tube 6, and an opening is provided at the other end portion 6f on the axial-directionally opposite side to the one end portion 6b. In the case of a prefilled syringe, a tube hole 6a of the outer tube 6 is filled with a drug, and a gasket of a plunger is inserted and plugged from the other side in the axial direction of the outer tube 6.

In addition, the outer tube 6 is provided with a holding hole 6c at the one end portion 6b in the axial direction thereof. The holding hole 6c is opened in a tapered shape such that its diameter increases along the direction from the other side toward the one side in the axial direction of the outer tube 6. The proximal section 3b as the other end in the axial direction of the needle tube 3 is inserted into the holding hole 6c (see FIG. 4C). With the proximal section 3b of the needle tube 3 inserted in the holding hole 6c, the tube hole 6a of the outer tube 6 and the needle tube 3 communicate with each other.

Besides, as shown in FIG. 4C, the needle holding member 7 is welded to that end face 6d of the one end portion 6b which is on the side opposite to the tube hole 6a side, the outer tube 6 and the needle holding member 7 are integrally joined to each other. The end face 6d is provided with a recess 6e to be engaged with a projection 8b of the needle holding member 7 which will be described later. The recess 6e is recessed from the end face 6d toward the other side in the axial direction.

Incidentally, while an example wherein the outer tube 6 of the syringe 2 is substantially cylindrical in shape has been described in the present embodiment, the shape of the outer tube 6 may be a hollow tetragonal prism or a hollow hexagonal prism.

As the material of the outer tube 6 configured as above, one of various resins is used. Examples of the resins include polyvinyl chloride, polyethylene, polypropylene, cyclic polyolefins, polystyrene, poly(4-methylpentene-1), polycarbonate, acrylic resin, acrylonitrile-butadiene-styrene copolymer, polyesters such as polyethylene terephthalate, etc., butadiene-styrene copolymer, and polyamides (e.g., nylon 6, nylon 6.6, nylon 6.10, nylon 12). Among these, such resins as polypropylene, cyclic polyolefins, polyesters, and poly(4-methylpentene-1) are preferably used. Incidentally, the material of the outer tube 6 is preferably transparent, for securing inside visibility.

The drug to be filled into the tube hole 6a of the outer tube 6 may be any of those drugs which are commonly used as injections. Examples of the drug include protein drugs such as antibodies, etc., peptide drugs such as hormones, etc., nucleic acid drugs, cell drugs, blood preparations, vaccines for prevention of various infectious diseases, carcinostatic agents, anesthetics, narcotic drugs, antibiotics, steroid preparations, proteinase inhibitors, heparin, saccharide injections such as glucose, etc., electrolyte correction injections such as sodium chloride, potassium lactate, etc., vitamin preparations, lipid emulsions, contrast media, and stimulants.

As shown in FIGS. 1 and 2, the needle holding member 7 is welded to one side in the axial direction of the outer tube 6. Examples of the method for welding the needle holding member 7 and the outer tube 6 to each other include ultrasonic welding and laser welding.

As shown in FIG. 4B, the needle holding member 7 has a distal end and a proximal end, and is formed integrally and coaxially with the needle tube 3, with the needle tip 3a of the needle tube 3 protruding from the distal end, by insert molding. On the axis of the needle holding member 7, the needle tube 3 is disposed along the axial direction.

In addition, the needle holding member 7 has a connecting part 8 to be joined to one axial-directional end of the outer tube 6, and a close contact part 9 continuous with the connecting section 8. As shown in FIGS. 1 and 2, the connecting part 8 is cross-shaped in section orthogonal to the axial direction of the needle holding member 7. The diameter of the connecting part 8 on the close contact part 9 side is smaller than the outside diameter of the close contact part 9. Therefore, the needle holding member 7 is shaped to be constricted at the portion of connection between the connecting part 8 and the close contact part 9.

Incidentally, the connecting part 8 is configured in such a way that, the farther the connecting part 8 goes away from the outer tube 6, the thinner the connecting part 8 becomes, in the present embodiment, however it may be continuous and remain constant in thickness.

Furthermore, the connecting part 8 is formed with a projection 8b at its contact surface 8a for contact with the end face 6d of the outer tube 6. This projection 8b is connected to the recess 6a provided in the end face 6d of the outer tube 6. With the projection 8b and the recess 6e connected to each other, the needle holding member 7 and the outer tube 6 are aligned. Furthermore, the projection 8b is melted by the heat generated at the time of welding of the needle holding member 7 and the outer tub 6 to each other. This ensures that bond properties between the needle holding member 7 and the outer tube 6 can be enhanced.

Incidentally, while an example wherein the outer tube 6 is provided with the recess 6e and the needle holding member 7 is provided with the projection 8b has been described in the present embodiment, a configuration may be adopted wherein the outer tube 6 is provided with a projection and the needle holding member 7 is provided with a recess for connection with the projection. Furthermore, a configuration may be adopted wherein the connecting part 8 of the needle holding member 7 is provided with an outer flange portion on the axial-directionally other side and the outer flange portion is provided with a projection or recess.

In addition, while an example wherein the connecting part 8 is cross-shaped for reducing the material thickness and securing strength has been described above, this is not restrictive. For example, the shape of the connecting part 8 may be a substantially cylindrical shape, a tetragonal prism, or a hexagonal prism.

The close contact part 9 is provided at one axial-directional end of the connecting part 8, or on the distal end of the needle holding member 7. The close contact part 9 is substantially cylindrical in shape. The needle tip 3a of the needle tube 3 protrudes from one side in the axial direction of the close contact part 9. In addition, an outer circumferential surface of the close contact part 9 is put into close contact with an inner circumferential surface of the cap 4 which will be described later.

Incidentally, while an example wherein the close contact part 9 is substantially cylindrical in shape has been described above, the shape of the close contact part 9 may be a tetragonal prism or a hexagonal prism, insofar as the shape corresponds to the shape of a tube hole 4a of the cap 4.

The needle holding member 7 configured as above is composed of essentially the same material as the outer tube 6. Therefore, at the time of welding the needle holding member 7 and the outer tube 6 to each other, good bond properties can be obtained, whereby the needle holding member 7 and the outer tube 6 can be firmly fixed to each other. In addition, the weld surface between the outer tube 6 and the needle holding member 7 can be made inconspicuous, so that enhanced appearance quality of the syringe 2 can be promised. Furthermore, with the syringe 2 divided into the outer tube 6 and the holding member 7, a mold for insert molding can be reduced in size.

### Cap

With reference to FIGS. 5 and 6, an embodiment of the cap 4 will be described below. FIG. 5 is a perspective view showing the state in which the cap 4 is attached to the syringe 1 provided with a needle, and FIG. 6 is a sectional view showing the syringe 1 provided with a needle and the cap 4 which are shown in FIG. 5.

As shown in FIGS. 5 and 6, the cap 4 is formed in a substantially cylindrical shape, with its one axial-directional end opening and with its other axial-directional end being closed. The cap 4 is formed, for example, from an elastic material, such as rubber or elastomer. In addition, as shown in FIG. 5, the cap 4 is attached to one side in the axial direction of the syringe 2 in such a manner as to cover the needle tip 3a of the needle tube ant the needle holding member 7 of the syringe 2. Besides, as shown in FIG. 6, the needle tip 3a side of the needle tube 3 and the needle holding member 7 are inserted in the tube hole 4a of the cap 4.

Incidentally, the inside diameter of the tube hole 4a of the cap 4 is set to be approximately equal to or slightly smaller than the outside diameter of the close contact part 9 of the needle holding member 7. Therefore, when the cap 4 is mounted to the syringe 2, the outer circumferential surface of the close contact part 9 of the needle holding member 7 makes close contact with the inner circumferential surface of the cap 4. This ensures that the needle tip 3a side of the needle tube 3, on the distal side relative to the close contact part 9 of the needle holding member 7, is hermetically sealed by the close contact part 9 and the inner circumferential surface of the cap 4. As a result, adhesion of bacteria to the needle tip 3a can be prevented.

In addition, the inner circumferential surface of the cap 4 fastens, by its elastic force, the portion of connection between the close contact part 9 and the connecting part 8 of the needle holding member 7, or the constricted portion. Consequently, the inner circumferential surface of the cap 4 and the constricted portion of the needle holding member 7 are engaged with each other, whereby the cap 4 can be prevented from being disengaged from the syringe 2 during transportation.

Assemblage of Syringe Provided with Needle

Now, a method for assembling the syringe 1 provided with a needle configured as above will be described below, referring to FIGS. 4A to 4C.

First, the needle tube 3 shown in FIG. 4A is formed with the needle holding member 7. Specifically, a resin is poured into a mold in which the needle tube is disposed, and insert molding is conducted thereby. As a result, as shown in FIG. 4B, the needle holding member 7 is formed around the needle tube 3, and the needle tube 3 is held by the needle holding member 7. In this instance, the needle tip 3a of the needle tube 3 protrudes from the distal end of the needle holding member 7, on one side in the axial direction of the close contact part 9, whereas the proximal section 3b of the needle tube 3 protrudes from the other side in the axial direction of the connecting part 8.

In addition, the outer tube 6 is molded separately from the needle tube 3 and the needle holding member 7, by use of a mold. With the outer tube 6 molded separately from the needle tube 3, a core pin for forming the tube hole 6a at the time of molding the outer tube 6 does not have to be provided with a portion for supporting the needle tube 3. As a result, as compared with a related-art syringe provided with a needle such that a needle tube and a syringe are formed at a time, dead volume of the tube hole 6a can be reduced and, therefore, the amount of the liquid drug left uninjected can be reduced. Besides, with the outer tube 6 and the needle holding member 7 molded separately from each other, a reduction in the size of the mold can be achieved.

Next, as shown in FIG. 4C, with the contact surface 8a of the needle holding member 7 and the end face 6d of the outer tube 6 kept in contact, the proximal section 3b of the needle tube 3 held by the needle holding member 7 is inserted into the holding hole 6c of the outer tube 6. Here, since the holding hole 6c is formed in a tapered shape, the proximal section 3b of the needle tube 3 can be easily inserted into the holding hole 6c, and assemblage of the syringe 1 provided with a needle can be carried out easily.

Furthermore, when the contact surface 8a of the needle holding member 7 and the end face 6d of the outer tube 6 are put in contact with each other, the projection 8b of the needle holding member 7 is inserted in the recess 6e provided in the outer tube 6. By this, alignment of the needle holding member 7 and the outer tube 6 can be achieved. Incidentally, since the positional matching between the needle holding member 7 and the outer tube 6 can be performed by only inserting the proximal section 3b of the needle tube 3 into the holding hole 6c, the object can be attained without providing the recess 6e and the projection 8b.

Next, contact surfaces of the outer tube 6 and the needle holding member 7 are welded to each other, whereby the outer tube 6 and the needle holding member 7 are joined to each other. In this instance, the projection 8b provided as a part of the needle holding member 7 is melted by the heat generated at the time of welding and penetrates into the recess 6e and into the space between the end face 6d of the outer tube 6 and the contact surface 8a of the needle holding member 7. In addition, since the outer tube 6 and the needle holding member 7 are formed from essentially the same material, good bond properties can be obtained. As a result, the needle holding member 7 and the outer tube 6 can be firmly fixed to each other, and assemblage of the syringe 1 provided with a needle is completed.

As above-mentioned, according to the syringe 1 provided with a needle of the present embodiment, the needle tube 3 and the needle holding member 7 are integrally formed by insert molding, and the needle holding member 7 which holds the needle tube 3 and the outer tube 6 are fixed by welding. Therefore, the syringe 1 provided with a needle can be assembled without using any adhesive. Accordingly, an adhering step to be conducted using an adhesive can be omitted. In addition, since no adhesive is used, there is no fear that an adhesive might make contact with the drug filled in the syringe 2, thereby adversely affecting the drug.

Incidentally, the present invention is not restricted to the embodiment described above and illustrated in the drawings, and various modifications are possible without departure from the gist of the invention as described in the claims.

1. A syringe provided with a needle, comprising:
a needle tube having a needle tip capable of puncturing a living body;
a needle holding member formed integrally and coaxially with the needle tube by insert molding, having a distal end and a proximal end, and configured to hold the needle tube such that the needle tip of the needle tube protrudes from the distal end; and
an outer tube having one end portion to which the proximal end of the needle holding member is fixed by welding, and another end portion located on the side axial-directionally opposite to the one end portion and provided with an opening, the outer tube being configured to be filled with a drug,
wherein the needle holding member and the outer tube are formed from essentially the same material.

2. The syringe provided with a needle according to claim 1,
wherein the one end portion of the outer tube is provided with a holding hole into which a proximal part of the needle tube is inserted, the proximal part being located on an opposite side to the needle tip.

3. The syringe provided with a needle according to claim 2,
wherein the holding hole is opened in a tapered shape such that a diameter thereof continuously increases along a direction from a side of the another end portion to a side of the one end portion of the outer tube.

4. The syringe provided with a needle according to claim 1,
wherein an end face of the one end portion of the outer tube, the needle holding member being fixed to the end face, is provided with a projection or recess, and
a contact surface of the needle holding member configured to make contact with the end face of the one end portion of the outer tube is provided with a recess or projection configured to be connected to the projection or recess of the end face.

5. A method for producing a syringe provided with a needle, comprising the steps of:
forming an outer tube configured to be filled with a drug;
integrally forming a needle tube having a needle tip capable of puncturing a living body with a needle holding member which holds the needle tube by an insert molding; and
fixing the needle holding member formed integrally with the needle tube to the outer tube by welding,
wherein the needle holding member and the outer tube are formed from essentially the same material.
